SEQUENCE LISTING (51 sequences)

SEQ ID NO: 1 - protein, 8 aa, Artificial Sequence
  Description of Artificial SequenceAII
DRVYIHPF

SEQ ID NO: 2 - protein, 7 aa, Artificial Sequence
  Description of Artificial SequenceAII (2-8)
RVYIHPF

SEQ ID NO: 3 - protein, 6 aa, Artificial Sequence
  Description of Artificial SequenceAII (3-8)
VYIHPF

SEQ ID NO: 4 - protein, 7 aa, Artificial Sequence
  Description of Artificial SequenceAII (1-7)
DRVYIHP

SEQ ID NO: 5 - protein, 6 aa, Artificial Sequence
  Description of Artificial SequenceAII (2-7)
RVYIHP

SEQ ID NO: 6 - protein, 5 aa, Artificial Sequence
  Description of Artificial SequenceAII (3-7)
VYIHP

SEQ ID NO: 7 - protein, 4 aa, Artificial Sequence
  Description of Artificial SequenceAII (5-8)
IHPF

SEQ ID NO: 8 - protein, 6 aa, Artificial Sequence
  Description of Artificial SequenceAII (1-6)
DRVYIH

SEQ ID NO: 9 - protein, 5 aa, Artificial Sequence
  Description of Artificial SequenceAII (1-5)
DRVYI

SEQ ID NO: 10 - protein, 4 aa, Artificial Sequence
  Description of Artificial SequenceAII (1-4)
DRVY

SEQ ID NO: 11 - protein, 3 aa, Artificial Sequence
  Description of Artificial SequenceAII (1-3)
DRV

SEQ ID NO: 12 - protein, 7 aa, Artificial Sequence
  Description of Artificial SequenceAII analogue
RXYIHPF

SEQ ID NO: 13 - protein, 7 aa, Artificial Sequence
  Description of Artificial SequenceAII analogue
RVYXHPF

SEQ ID NO: 14 - protein, 3 aa, Artificial Sequence
  Description of Artificial SequenceAII (6-8)
HPF

SEQ ID NO: 15 - protein, 5 aa, Artificial Sequence
  Description of Artificial SequenceAII (4-8)
YIHPF

SEQ ID NO: 16 - protein, 7 aa, Artificial Sequence
  Description of Artificial SequenceAII analogue class
XXYXHPF

SEQ ID NO: 17 - protein, 7 aa, Artificial Sequence
  Description of Artificial SequenceAII analogue
RVYGHPF

SEQ ID NO: 18 - protein, 7 aa, Artificial Sequence
  Description of Artificial SequenceAII analogue
RVYAHPF

SEQ ID NO: 19 - protein, 8 aa, Artificial Sequence
  Description of Artificial SequenceAII analogue 1
DRVYVHPF

SEQ ID NO: 20 - protein, 8 aa, Artificial Sequence
  Description of Artificial SequenceAII analogue 2
NRVYVHPF

SEQ ID NO: 21 - protein, 11 aa, Artificial Sequence
  Description of Artificial SequenceAII analogue 3
APGDRIYVHPF

SEQ ID NO: 22 - protein, 8 aa, Artificial Sequence
  Description of Artificial SequenceAII analogue 4
ERVYIHPF

SEQ ID NO: 23 - protein, 8 aa, Artificial Sequence
  Description of Artificial SequenceAII analogue 5
DKVYIHPF

SEQ ID NO: 24 - protein, 8 aa, Artificial Sequence
  Description of Artificial SequenceAII analogue 6
DRAYIHPF

SEQ ID NO: 25 - protein, 8 aa, Artificial Sequence
  Description of Artificial SequenceAII analogue 7
DRVTIHPF

SEQ ID NO: 26 - protein, 8 aa, Artificial Sequence
  Description of Artificial SequenceAII analogue 8
DRVYLHPF

SEQ ID NO: 27 - protein, 8 aa, Artificial Sequence
  Description of Artificial SequenceAII analogue 9
DRVYIRPF

SEQ ID NO: 28 - protein, 8 aa, Artificial Sequence
  Description of Artificial SequenceAII analogue 10
DRVYIHAF

SEQ ID NO: 29 - protein, 8 aa, Artificial Sequence
  Description of Artificial SequenceAII analogue 11
DRVYIHPY

SEQ ID NO: 30 - protein, 8 aa, Artificial Sequence
  Description of Artificial SequenceAII analogue 12
PRVYIHPF

SEQ ID NO: 31 - protein, 8 aa, Artificial Sequence
  Description of Artificial SequenceAII analogue 13
DRPYIHPF

SEQ ID NO: 32 - protein, 8 aa, Artificial Sequence
  Description of Artificial SequenceAII analogue 14
DRVYIHPF

SEQ ID NO: 33 - protein, 8 aa, Artificial Sequence
  Description of Artificial SequenceAII analogue 15
DRXYIHPF

SEQ ID NO: 34 - protein, 8 aa, Artificial Sequence
  Description of Artificial SequenceAII analogue 16
DRVYXHPF

SEQ ID NO: 35 - protein, 9 aa, Artificial Sequence
  Description of Artificial SequenceAII analogue 17
DRVSYIHPF

SEQ ID NO: 36 - protein, 8 aa, Artificial Sequence
  Description of Artificial Sequencep-aminophenylalanine 6 AII
DRVYIXPF

SEQ ID NO: 37 - protein, 10 aa, Artificial Sequence
  Description of Artificial Sequenceangiotensin I
DRVYIHPFHL

SEQ ID NO: 38 - protein, 7 aa, Artificial Sequence
  Description of Artificial Sequence 1GDAla4-AII(1-7)
DRVAIHP

SEQ ID NO: 39 - protein, 7 aa, Artificial Sequence
  Description of Artificial Sequence 2GD Pro3-AII(1-7)
DRPYIHP

SEQ ID NO: 40 - protein, 7 aa, Artificial Sequence
  Description of Artificial Sequence 5GD Lys 3-AII(1-7)
DRKYIHP

SEQ ID NO: 41 - protein, 7 aa, Artificial Sequence
  Description of Artificial Sequence 9GD Norleu-AII(1-7)
DRXYIHP

SEQ ID NO: 42 - protein, 8 aa, Artificial Sequence
  Description of Artificial Sequence GSD28 Ile8-AII
DRVYIHPI

SEQ ID NO: 43 - protein, 8 aa, Artificial Sequence
  Description of Artificial Sequence Ala3aminoPhe6-AII
DRAYIXPF

SEQ ID NO: 44 - protein, 7 aa, Artificial Sequence
  Description of Artificial Sequence Ala3-AIII
RVAIHPF

SEQ ID NO: 45 - protein, 8 aa, Artificial Sequence
  Description of Artificial SequenceGly1-AII
GRVYIHPF

SEQ ID NO: 46 - protein, 8 aa, Artificial Sequence
  MOD_RES (4): Nle
RVYXLHPF

SEQ ID NO: 47 - protein, 8 aa, Artificial Sequence
  Description of Artificial Sequence Acpc3-AII
DRXYIHPF

SEQ ID NO: 48 - protein, 8 aa, Artificial Sequence
  Description of Artificial Sequence Orn2-AII
DXVYIHPF

SEQ ID NO: 49 - protein, 8 aa, Artificial Sequence
  Description of Artificial Sequence Citron2-AII
DXVYIHPF

SEQ ID NO: 50 - protein, 7 aa, Artificial Sequence
  Description of Artificial Sequence Pro3Ala4-AII(1-7)
DRPAIHP

SEQ ID NO: 51 - protein, 7 aa, Artificial Sequence
  Description of Artificial Sequence HydroxyPro3-AII(1-7)
DRXYIHP

(19) United States
(12) Patent Application Publication
(10) Pub. No.: US 2002/0049162 A1
(43) Pub. Date: Apr. 25, 2002

(54) Methods for inhibiting smooth muscle cell proliferation

(76) Inventors: Kathleen E. Rodgers, Long Beach, CA (US); Gere S. DiZerega, Los Angeles, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/771,192

(22) Filed: Jan. 26, 2001

Related U.S. Application Data
(60) Provisional application No. 60/178,423, filed on Jan. 27, 2000.

(51) Int. Cl. A61K 38/08

(52) U.S. Cl. 514/16

(57) ABSTRACT

The present invention provides improved methods and pharmaceutical compositions for inhibiting smooth muscle cell proliferation, and for treating and preventing conditions associated with smooth muscle cell proliferation, by administering an effective amount of angiotensinogen, angiotensin I (AI), AI analogues, AI fragments and analogues thereof, angiotensin II (AII) analogues, AII fragments or analogues thereof or AII AT₂ type 2 receptor agonists to the subject.

### CROSS-REFERENCE

This application claims priority to U.S. Patent application Ser. No. 60/178,423 filed Jan. 27, 2000.

### FIELD OF THE INVENTION

This present invention relates to methods to inhibit smooth muscle cell proliferation and to treat and prevent conditions associated with smooth muscle cell proliferation.

### BACKGROUND OF THE INVENTION

Smooth muscle cells perform diverse functions in multiple tissues including the arterial wall, uterus, respiratory, urinary and digestive tracts, due in large part to their phenotypic plasticity. In contrast to skeletal muscle cells, smooth muscle cells are capable of simultaneously proliferating and expressing a set of lineage-restricted proteins including myofibrillar isoforms, cell surface receptors and smooth muscle cell-restricted enzymes. (U.S. Pat. No. 6,114,311)

Many pathological conditions have been found to be associated with smooth muscle cell proliferation. Such conditions particularly include restenosis and atherosclerosis. (U.S. Pat. Nos. 6,166,090 and 6,114,311)

For example, restenosis occurs after a variety of arterial insults such as angioplasty, stent placement, bypass surgery, heart transplantation and endarterectomy. (U.S. Pat. No. 5,409,926; incorporated by reference herein in its entirety.) Each of these procedures is important for the treatment of coronary artery disease. Despite initial success and limited complications using these procedures, restenosis occurs at the site of treatment in a significant number of patients between one and six months after treatment. Restenosis continues to be the main complication associated with these procedures, resulting in significant morbidity and mortality, and frequently necessitates further procedures. (U.S. Pat. No. 5,981,568; incorporated by reference herein in its entirety.) The processes responsible for restenosis are not completely understood, but may result from a complex interplay among several different biologic agents and pathways. (U.S. Pat. No. 5,981,568; incorporated by reference herein in its entirety.) Viewed in histological sections, restenotic lesions may have an overgrowth of smooth muscle cells in the intimal layers of the vessel (Johnson et al., Circulation, 78 (Suppl. II): II-82 (1988)). Several possible mechanisms for smooth muscle cell proliferation after angioplasty have been suggested (Popma et al., Amer. J. Med., 88: 16N-24N (1990); Fanelli et al, Amer. Heart Jour., 119: 357-368 (1990); Liu et al., Circulation, 79: 1374-1387 (1989); Clowes et al., Circ. Res., 56: 139-145 (1985)).

Various drugs have been used in an attempt to prevent restenosis, including anticoagulants such as heparin, platelet aggregation inhibitors such as aspirin, dipyridamole, ticlopidine, prostacyclin and its derivatives, cell proliferation inhibitors, corticosteroids, calcium channel blockers, angiotensin converting enzyme inhibitors, angiotensin II receptor antagonists, and antilipidemics such as eicosapentaenoic acid, and lovastatin, but none of them proved to be sufficiently effective from the clinical viewpoint. (U.S. Pat. Nos. 5,993,797 and 5,409,926; Wilson et al., Cardiovascular Res. 42(3):761-772 (1999); Angiology 50(10):811-822 (1999); all references incorporated by reference herein in their entirety.)

Thus, there is a need in the art for other methods of treating and preventing conditions associated with smooth muscle cell proliferation, including but not limited to restenosis.

### SUMMARY OF THE INVENTION

The present invention provides methods and kits for inhibiting smooth muscle cell proliferation, and for treating and preventing conditions associated with smooth muscle cell proliferation, by administering to a patient in need thereof an amount effective of angiotensinogen, angiotensin I (AI), AI analogues, AI fragments and analogues thereof, angiotensin II analogues, AII fragments or analogues thereof or AII AT₂ type 2 receptor agonists, either alone, combined, or in further combination with other compounds, for treating or preventing restenosis, such as anticoagulants, platelet aggregation inhibitors, smooth muscle cell proliferation inhibitors, calcium channel blockers, angiotensin converting enzyme inhibitors, angiotensin II receptor antagonists, and antilipidemics.

### DETAILED DESCRIPTION OF THE PREFERRED EMBODIMENTS

Unless otherwise indicated, the term “angiotensin converting enzyme inhibitors” or “ACE inhibitors” includes any compound that inhibits the conversion of the decapeptide angiotensin I to angiotensin II, and include but are not limited to alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. (See for example Jackson, et al., Renin and Angiotensin in Goodman & Gilman's The Pharmacological Basis of Therapeutics, 9th ed., eds. Hardman, et al. (McGraw Hill, 1996); and U.S. Pat. No. 5,977,159.)

Unless otherwise indicated, the term “active agents” as used herein refers to the group of compounds comprising angiotensinogen, angiotensin I (AI), AI analogues, AI fragments and analogues thereof, angiotensin II (AII) analogues, AII fragments or analogues thereof or AII AT₂ type 2 receptor agonists, either alone, combined, or in further combination with other compounds, for inhibiting smooth muscle cell proliferation, and for treating or preventing conditions associated with smooth muscle cell proliferation, such as anticoagulants, platelet aggregation inhibitors, smooth muscle cell proliferation inhibitors, calcium channel blockers, angiotensin converting enzyme inhibitors, angiotensin II receptor antagonists, and antilipidemics.

As used herein, the term “conditions associated with smooth muscle cell proliferation” include, but are not limited to restenosis, atherosclerosis, and other disorders characterized by excessive and/or aberrant smooth muscle cell proliferation.

As defined herein, the term “restenosis” encompasses any diminution in vessel lumen volume, area and/or diameter associated with a procedural vascular trauma.

As used herein, “vessels” includes mammalian vessels, e.g., coronary vessels as well as peripheral, femoral and carotid vessels.

As used herein, “vascular trauma” includes but is not limited to trauma associated with an interventional procedure, such as angioplasty, placement of a stent, shunt, stet, synthetic or natural graft, adventitial wrap, indwelling catheter or other implantable devices. Grafts include synthetic active agent-treated grafts, e.g., impregnated or coated grafts.

The methods of the present invention find use in any patient with a condition associated with excessive and/or aberrant smooth muscle cell proliferation. For example, the method is of substantial value to a patient undergoing or who has undergone any procedure in which restenosis is a concern, including but not limited to angioplasty, stent placement, bypass surgery, heart transplantation and endarterectomy. The active agents are preferably administered to the patient at the time of the procedure or as soon as possible thereafter in order to prevent or ameliorate restenosis.

U.S. Pat. No. 5,015,629 to DiZerega (the entire disclosure of which is hereby incorporated by reference) describes a method for increasing the rate of healing of wound tissue, comprising the application to such tissue of angiotensin II (AII) in an amount which is sufficient for said increase. The application of AII to wound tissue significantly increases the rate of wound healing, leading to a more rapid re-epithelialization and tissue repair. The term AII refers to an octapeptide present in humans and other species having the sequence Asp—Arg—Val—Tyr—Ile—His—Pro—Phe [SEQ ID NO: 1]. The biological formation of angiotensin is initiated by the action of renin on the plasma substrate angiotensinogen (Circulation Research 60:786-790 (1987); Clouston et al., Genomics 2:240-248 (1988); Kageyama et al., Biochemistry 23:3603-3609; Ohkubo et al,, Proc. Natl. Acad. Sci. 80:2196-2200 (1983)); all references hereby incorporated in their entirety). The substance so formed is a decapeptide called angiotensin I (AI) which is converted to AII by the converting enzyme angiotensinase which removes the C-terminal His—Leu residues from AI, Asp—Arg—Val—Tyr—Ile—His—Pro—Phe—His—Leu [SEQ ID NO:37]. AII is a known pressor agent and is commercially available.

Studies have shown that AII increases mitogenesis and chemotaxis in cultured cells that are involved in wound repair, and also increases their release of growth factors and extracellular matrices (dizerega, U.S. Pat. No. 5,015,629; Dzau et. al., J. Mol. Cell. Cardiol. 21:S7 (Supp III) 1989; Berk et. al., Hypertension 13:305-14 (1989); Kawahara, et al., BBRC 150:52-9 (1988); Naftilan, et al., J. Clin. Invest. 83:1419-23 (1989); Taubman et al., J. Biol. Chem. 264:526-530 (1989); Nakahara, et al., BBRC 184:811-8 (1992); Stouffer and Owens, Circ. Res. 70:820 (1992); Wolf, et al., Am. J Pathol. 140:95-107 (1992); Bell and Madri, Am. J. Pathol. 137:7-12 (1990)). In addition, AII was shown to be angiogenic in rabbit corneal eye and chick chorioallantoic membrane models (Fernandez, et al., J. Lab. Clin. Med. 105:141 (1985); LeNoble, et al., Eur. J. Pharmacol. 195:305-6 (1991)).

We have previously demonstrated that angiotensinogen, angiotensin I (AI), AI analogues, AI fragments and analogues thereof, angiotensin II (AII), AII analogues, AII fragments or analogues thereof; AII AT₂ type 2 receptor agonists are effective in accelerating wound healing and the proliferation of certain cell types. See, for example, co-pending U.S. Patent application Ser. Nos. 08/126,370, Filed Sep. 24, 1993; 09/208,337, Filed Dec. 9,1998; 09/108,478, 09/434,746 filed Nov. 5, 1999; Filed Jun. 30, 1998; 09/503,872, Feb. 14, 2000; 08/990,664, Dec. 15, 1997; 09/210,249, Dec. 11, 1998; 09/098,806, Nov. 24, 1998; 09/012,400, Jan. 23, 1998; 09/264,563, Mar. 8, 1999; 09/287,674, Apr. 7, 1999; 09/307,940, May 10, 1999; 09/246,162, Feb. 8, 1999; 09/255,136, Feb. 19, 1999; 09/245,680, Feb. 8, 1999; 09/250,703, Feb. 15, 1999; 09/246,525, Feb. 8, 1999; 09/266,293, Mar. 11, 1999; 09/332,582, Jun. 14, 1999; 09/373,962, Aug. 13, 1999; 09/352,191, Jul. 12, 1999; as well as U.S. Pat. Ser. Nos. 5,015,629; 5,629,292; 5,716,935; 5,834,432; and 5,955,430; 6,096,709; 6,110,895.

The effect of AII on a given cell type has been hypothesized to be dependent, in part, upon the AII receptor subtypes the cell expresses (Shanugam et al., Am. J. Physiol. 268:F922-F930 (1995); Helin et al., Annals of Medicine 29:23-29 (1997); Bedecs et al., Biochem J. 325:449-454 (1997)). These studies have shown that AII receptor subtype expression is a dynamic process that changes during development, at least in some cell types. AII activity is typically modulated by either or both the AT1 and AT2 AII receptors. However, AII has recently been shown to stimulate proliferation of primary human keratinocytes via a non-AT1, non-AT2 receptor. (Steckelings et al., Biochem. Biophys. Res. Commun. 229:329-333 (1996)). These results underscore the cell-type (ie: based on receptor expression) specific nature of AII activity.

Many studies have focused upon AII(1-7) (AII residues 1-7) (SEQ ID NO:4) or other fragments of AII to evaluate their activity. AII(1-7) elicits some, but not the full range of effects elicited by AII. (Pfeilschifter, et al., Eur. J. Pharmacol. 225:57-62 (1992); Jaiswal, et al., Hypertension 19(Supp. II):II-49-II-55 (1992); Edwards and Stack, J. Pharmacol. Exper. Ther. 266:506-510 (1993); Jaiswal, et al., J. Pharmacol. Exper. Ther. 265:664-673 (1991); Jaiswal, et al., Hypertension 17:1115-1120 (1991); Portsi, et a., Br. J. Pharmacol. 111:652-654 (1994)).

Other data suggests that the AI fragment AII(1-7) acts through a receptor(s) that is distinct from the AT1 and AT2 receptors which modulate AII activity. (Ferrario et al., J. Am. Soc. Nephrol. 9:1716-1722 (1998); Iyer et al., Hypertension 31:699-705 (1998); Freeman et al., Hypertension 28:104 (1996); Ambuhl et al., Brain Res. Bull. 35:289 (1994)). Thus, AII(1-7) activity on a particular cell type cannot be predicted based solely on the effect of AII on the same cell type. In fact, there is some evidence that AII(1-7) often opposes the actions of AII. (See, for example, Ferrario et al., Hypertension 30:535-541 (1997)).

AII(1-7) (SEQ ID NO:4) has been shown to inhibit smooth muscle cell proliferation, and to reduce smooth muscle cell growth after vascular injury. (Strawn et al., Hypertension 33:207-211 (1999)). ACE inhibitors have been suggested for the treatment of restenosis. (See, for example, Okamura et al., Angiology 50:811-822 (1999)) The use of AII receptor antagonists has also been suggested for the treatment of restenosis. (See for example Wilson et al., Cardiovasc. Res. 42:761-772 (1999); Griendling et al., Annual Rev. Pharmacol. Toxicol. 36:281-306 (1996)) AII is believed to be involved in the pathogenesis of restenosis, due in part to its stimulatory effect on smooth musclce cell proliferation. (See, for example Strawn et al., 1999).

Based on the above, there would be no expectation by one of skill in the art that angiotensinogen, angiotensin I (AI), AI analogues, AI fragments and analogues thereof, angiotensin II (AII) analogues, AII fragments or analogues thereof or AII AT₂ type 2 receptor agonists could be used to treat and prevent smooth muscle cell proliferation and to treat or prevent conditions associated with smooth muscle cell proliferation.

A peptide agonist selective for the AT2 receptor (AII has 100 times higher affinity for AT2 than AT1) is p-aminophenylalanine6-AII [“(P-NH₂-Phe)6-AII)”], Asp—Arg—Val—Tyr—Ile—Xaa—Pro—Phe [SEQ ID NO.36] wherein Xaa is p-NH₂-Phe (Speth and Kim, BBRC 169:997-1006 (1990). This peptide gave binding characteristics comparable to AT2 antagonists in the experimental models tested (Catalioto, et al., Eur. J. Pharmacol. 256:93-97 (1994); Bryson, et al., Eur. J. Pharmacol. 225:119-127 (1992).

The effects of AII and AII receptor antagonists have been examined in two experimental models of vascular injury and repair which suggest that both AII receptor subtypes (AT1 and AT2) play a role in wound healing (Janiak et al., Hypertension 20:737-45 (1992); Prescott, et al., Am. J. Pathol. 139:1291-1296 (1991); Kauffman, et al., Life Sci. 49:223-228 (1991); Viswanathan, et al., Peptides 13:783-786 (1992); Kimura, et al., BBRC 187:1083-1090 (1992)).

As hereinafter defined, a preferred class of AT2 agonists for use in accordance with the present invention comprises AII analogues or active fragments thereof having p-NH-Phe in a position corresponding to a position 6 of AII. In addition to peptide agents, various nonpeptidic agents (e.g., peptidomimetics) having the requisite AT2 agonist activity are further contemplated for use in accordance with the present invention.

The active AII analogues, fragments of AII and analogues thereof of particular interest in accordance with the present invention comprise a sequence of at least three contiguous amino acids of groups R¹—R⁸ in the sequence of general formula I

R¹—R²—R³—R⁴—R⁵—R⁶—R⁷—R⁸

wherein R¹ is selected from the group consisting of H, Asp, Glu, Asn, Acpc (1-aminocyclopentane carboxylic acid), AIa, Me²Gly, Pro, Bet, Glu(NH₂), Gly, Asp(NH₂) and Suc,

R² is is selected from the group consisting of Arg, Lys, AIa, Citron, Orn, Ser(Ac), Sar, D-Arg and D-Lys,

R³ is selected from the group consisting of Val, AIa, Leu, norLeu, Ile, Gly, Lys, Pro, HydroxyPro, Aib, Acpc and Tyr;

R⁴ is selected from the group consisting of Tyr, Tyr(PO₃)₂, Thr, Ser, homoSer, azaTyr, and AIa;

R⁵ is selected from the group consisting of Ile, AIa, Leu, norLeu, Val and Gly;

R⁶ is selected from the group consisting of His, Arg or 6-NH₂-Phe;

R⁷ is selected from the group consisting of Pro or AIa; and

R⁸ is selected from the group consisting of Phe, Phe(Br), Ile and Tyr, excluding sequences including R⁴ as a terminal Tyr group, and wherein the active agent does not consist essentially of SEQ ID NO:1, SEQ ID NO:2, or SEQ ID NO:4.

In alternate embodiments, the active agents comprise a sequence of at least four, five, six, or seven contiguous amino acids of groups R¹—R⁸ in the sequence of general formula I. In a further alternative, the active agents consist essentially of a sequence of at least four, five, six, or seven contiguous amino acids of groups R¹—R⁸ in the sequence of general formula I.

Compounds falling within the category of AT2 agonists useful in the practice of the invention include the AII analogues set forth above subject to the restriction that R⁶ is p-NH₂-Phe.

Particularly preferred combinations for R^{A} and R^{B} are Asp—Arg, Asp—Lys, Glu—Arg and Glu—Lys. Particularly preferred embodiments of this class include the following: AII(3-8), also known as desl-AIII or AIV, Val—Tyr—Ile—His—Pro—Phe [SEQ ID NO:3]; AII(2-7). Arg—Val—Tyr—Ile—His—Pro [SEQ ID NO:5]; AII(3-7), Val—Tyr—Ile—His—Pro [SEQ ID NO:6]; AII(5-8), Ile—His—Pro—Phe [SEQ ID NO:7]; AII(1-6), Asp—Arg—Val—Tyr—Ile—His [SEQ ID NO:8]; AII(1-5), Asp—Arg—Val—Tyr—Ile [SEQ ID NO:9]; AII(1-4), Asp—Arg—Val—Tyr [SEQ ID NO:10]; and AII(1-3), Asp—Arg—Val [SEQ ID NO:11]. Other preferred embodiments include: Arg—norLeu—Tyr—Ile—His—Pro—Phe [SEQ ID NO:12] and Arg—Val—Tyr—norLeu—His—Pro—Phe [SEQ ID NO:13]. Still another preferred embodiment encompassed within the scope of the invention is a peptide having the sequence Asp—Arg—Pro—Tyr—Ile—His—Pro—Phe [SEQ ID NO:31]. AII(6-8), His—Pro—Phe [SEQ ID NO:14] and AII(4-8), Tyr—Ile—His—Pro—Phe [SEQ ID NO:15] were also tested and found not to be effective.

Another class of compounds of particular interest in accordance with the present invention are those of the general formula II

R²—R³—R⁴—R⁵—R⁶—R⁷—R⁸

in which R² is selected from the group consisting of H, Arg, Lys, AIa, Om, Citron, Ser(Ac), Sar, D-Arg and D-Lys;

R³ is selected from the group consisting of Val, AIa, Leu, norLeu, Ile, Gly, Pro, Hydroxy-Pro, Aib, Acpc and Tyr;

R⁴ is selected from the group consisting of Tyr, Tyr(PO₃)₂, Thr, Ser, homoSer, azaTyr, and AIa;

R⁵ is selected from the group consisting of Ile, AIa, Leu, norLeu, Val and Gly;

R⁶ is His, Arg or 6-NH₂-Phe;

R⁷ is Pro or AIa; and

R⁸ is selected from the group consisting of Phe, Phe(Br), Ile and Tyr; wherein the active agent does not consist essentially of AII(2-8) (SEQ ID NO:2).

A particularly preferred subclass of the compounds of general formula II has the formula

R²—R³—Tyr—R⁵—His—Pro—Phe [SEQ ID NO:16]

wherein R², R³ and R⁵ are as previously defined. Particularly preferred compounds include peptides having the structures Arg—Val—Tyr—Gly—His—Pro—Phe [SEQ ID NO:17] and Arg—Val—Tyr—AIa—His—Pro—Phe [SEQ ID NO:18]. The fragment AII(4-8) was ineffective in repeated tests; this is believed to be due to the exposed tyrosine on the N-tenninus.

Other particularly preferred embodiments include:

| 1GD | Ala4-AII(1-7) | DRVAIHP | SEQ ID NO: 38 |
|---|---|---|---|
| 2GD | Pro3-AII(1-7) | DRPYIHP | SEQ ID NO: 39 |
| 5GD | Lys3-AII(1-7) | DRKYIHP | SEQ ID NO: 40 |
| 9GD | NorLeu-AII(1-7) | DR(nor)YIHP | SEQ ID NO: 41 |
| GSD 28 | Ile⁸-AII | DRVYIHPI | SEQ ID NO: 42 |
| | Ala3aminoPhe6 | DRAYIF*PF | SEQ ID NO: 43 |
| | AIII: | | |
| | Ala3-AIII | RVAIHPF | SEQ ID NO: 44 |
| | Gly¹-AII | GRVYIHPF | SEQ ID NO: 45 |
| | NorLeu⁴-AIII | --RVYnLHPF | SEQ ID NO: 46 |
| | Acpc³-AII | DR(Acpc)YIHPF | SEQ ID NO: 47 |
| GSD 37B | Orn²-AII | D(Orn)VYIHPF | SEQ ID NO: 48 |
| GSD38B | Citron²-AII | D(Citron)VYIHPF | SEQ ID NO: 49 |
| 3GD | Pro³Ala⁴-AII(1-7) | DRPAIHP | SEQ ID NO: 50 |
| 8GD | Hydroxy-Pro³-AII | DRP(OH)AIHP | SEQ ID NO: 51 |
| | (1-7) | | |

In the above formulas, the standard three-letter abbreviations for amino acid residues are employed. In the absence of an indication to the contrary, the L-form of the amino acid is intended. Other residues are abbreviated as follows:

TABLE 1
Abbreviation for Amino Acids

| | |
|---|---|
| Me²Gly | N,N-dimethylglycyl |
| Bet | 1-carboxy-N,N,N-trimethylmethanaminium hydroxide inner |
| | salt (betaine) |
| Suc | Succinyl |
| Phe(Br) | p-bromo-L-phenylalanyl |
| azaTyr | aza-α′-homo-L-tyrosyl |
| Acpc | 1-aminocyclopentane carboxylic acid |
| Aib | 2-aminoisobutyric acid |
| Sar | N-methylglycyl (sarcosine) |
| Cit | Citron |
| Orn | Ornithine |

It has been suggested that AII and its analogues adopt either a gamma or a beta turn (Regoli, et al., Pharmacological Reviews 26:69 (1974). In general, it is believed that neutral side chains in position R³, R⁵ and R⁷ may be involved in maintaining the appropriate distance between active groups in positions R⁴, R⁶ and R⁸ primarily responsible for binding to receptors and/or intrinsic activity. Hydrophobic side chains in positions R³, R⁵ and R⁸ may also play an important role in the whole conformation of the peptide and/or contribute to the formation of a hypothetical hydrophobic pocket.

Appropriate side chains on the amino acid in position R² may contribute to affinity of the compounds for target receptors and/or play an important role in the conformation of the peptide. For this reason, Arg and Lys are particularly preferred as R². AIternatively, R₂ may be H, AIa, Orn, Citron, Ser(Ac), Sar, D-Arg, or D-Lys.

For purposes of the present invention, it is believed that R³ may be involved in the formation of linear or nonlinear hydrogen bonds with R⁵ (in the gamma turn model) or R⁶ (in the beta turn model). R³ would also participate in the first turn in a beta antiparallel structure (which has also been proposed as a possible structure). In contrast to other positions in general formula I, it appears that beta and gamma branching are equally effective in this position. Moreover, a single hydrogen bond may be sufficient to maintain a relatively stable conformation. Accordingly, R³ may suitably be selected from Lys, Val, AIa, Leu, norLeu, Ile, Gly, Pro, Hydroxy—Pro, Aib, Acpc and Tyr.

With respect to R⁴, conformational analyses have suggested that the side chain in this position (as well as in R³ and R⁵) contribute to a hydrophobic cluster believed to be essential for occupation and stimulation of receptors. Thus, R⁴ is preferably selected from Tyr, Thr, Tyr (PO₃)₂, homoSer, Ser and azaTyr. In this position, Tyr is particularly preferred as it may form a hydrogen bond with the receptor site capable of accepting a hydrogen from the phenolic hydroxyl (Regoli, et al. (1974), supra). It has also been found that R⁴ can be AIa.

In position R⁵, an amino acid with a β aliphatic or alicyclic chain is particularly desirable. Therefore, while Gly is suitable in position R⁵, it is preferred that the amino acid in this position be selected from Ile, AIa, Leu, norLeu, and Val.

In the angiotensinogen, AI, AI analogues, AI fragments and analogues thereof, 5 AII analogues, fragments and analogues of fragments of particular interest in accordance with the present invention, R⁶ is His, Arg or 6-NH₂-Phe. The unique properties of the imidazole ring of histidine (e.g., ionization at physiological pH, ability to act as proton donor or acceptor, aromatic character) are believed to contribute to its particular utility as R⁶. For example, conformational models suggest that His may participate in hydrogen bond formation (in the beta model) or in the second turn of the antiparallel structure by influencing the orientation of R⁷. Similarly, it is presently considered that R⁷ should be Pro or AIa in order to provide the most desirable orientation of R⁸. In position R⁸, both a hydrophobic ring and an anionic carboxyl terminal appear to be particularly useful in binding of the analogues of interest to receptors; therefore, Tyr, Ile, Phe(Br), and especially Phe are preferred for purposes of the present invention.

Other active agents of particular interest include the following:

TABLE 2
Angiotensin II Analogues

| AII | | |
|---|---|---|
| Analogue | | Sequence |
| Name | Amino Acid Sequence | Identifier |
| Analogue 1 | Asp—Arg—Val—Tyr—Val—His—Pro—Phe | SEQ ID |
| | | NO: 19 |
| Analogue 2 | Asn—Arg—Val—Tyr—Val—His—Pro—Phe | SEQ ID |
| | | NO: 20 |
| Analogue 3 | Ala—Pro—Gly—Asp—Arg—Ile—Tyr— | SEQ ID |
| | Val—His—Pro—Phe | NO: 21 |
| Analogue 4 | Glu—Arg—Val—Tyr—Ile—His—Pro—Phe | SEQ ID |
| | | NO: 22 |
| Analogue 5 | Asp—Lys—Val—Tyr—Ile—His—Pro—Phe | SEQ ID |
| | | NO: 23 |
| Analogue 6 | Asp—Arg—Ala—Tyr—Ile—His—Pro—Phe | SEQ ID |
| | | NO: 24 |
| Analogue 7 | Asp—Arg—Val—Thr—Ile—His—Pro—Phe | SEQ ID |
| | | NO: 25 |
| Analogue 8 | Asp—Arg—Val—Tyr—Leu—His—Pro—Phe | SEQ ID |
| | | NO: 26 |
| Analogue 9 | Asp—Arg—Val—Tyr—Ile—Arg—Pro—Phe | SEQ ID |
| | | NO: 27 |
| Analogue 10 | Asp—Arg—Val—Tyr—Ile—His—Ala—Phe | SEQ ID |
| | | NO: 28 |
| Analogue 11 | Asp—Arg—Val—Tyr—Ile—His—Pro—Tyr | SEQ ID |
| | | NO: 29 |
| Analogue 12 | Pro—Arg—Val—Tyr—Ile—His—Pro—Phe | SEQ ID |
| | | NO: 30 |
| Analogue 13 | Asp—Arg—Pro—Tyr—Ile—His—Pro—Phe | SE ID |
| | | NO: 31 |
| Analogue 14 | Asp—Arg—Val—Tyr(PO₃)₂-Ile—His— | SEQ ID |
| | Pro—Phe | NO: 32 |
| Analogue 15 | Asp—Arg—norLeu—Tyr—Ile—His— | SEQ ID |
| | Pro—Phe | NO: 33 |
| Analogue 16 | Asp—Arg—Val—Tyr-norLeu—His— | SEQ ID |
| | Pro—Phe | NO: 34 |
| Analogue 17 | Asp—Arg—Val-homoSer—Tyr—Ile—His— | SEQ ID |
| | Pro—Phe | NO: 35 |

The polypeptides of the instant invention may be synthesized by any conventional method, including, but not limited to, those set forth in J. M. Stewart and J. D. Young, Solid Phase Peptide Synthesis, 2nd ed., Pierce Chemical Co., Rockford, Ill. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis. The disclosures of the foregoing treatises are incorporated by reference herein.

In general, these methods involve the sequential addition of protected amino acids 10 to a growing peptide chain (U.S. Pat. No. 5,693,616, herein incorporated by reference in its entirety). Normally, either the amino or carboxyl group of the first amino acid and any reactive side chain group are protected. This protected amino acid is then either attached to an inert solid support, or utilized in solution, and the next amino acid in the sequence, also suitably protected, is added under conditions amenable to formation of the amide linkage. After all the desired amino acids have been linked in the proper sequence, protecting groups and any solid support are removed to afford the crude polypeptide. The polypeptide is desalted and purified, preferably chromatographically, to yield the final product.

Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art.

AIternatively, the peptides can be produced by standard molecular biological techniques.

In one aspect of the present invention, a method of inhibiting smooth muscle cell proliferation is provided by contacting smooth muscle cells (SMCs) with an amount effective of the active agents of the present invention to inhibit SMC proliferation.

In another embodiment, the present invention provides methods for treating or preventing a condition associated with smooth muscle cell proliferation by administering to a patient in need thereof an amount effective to treat or prevent the condition of one or more of the active agents of the invention, either alone or in further combination with other compounds effective for treating or preventing conditions associated with smooth muscle cell proliferation. For examples, compounds useful for treating or preventing restenosis include, but are not limited to, anticoagulants such as heparin, platelet aggregation inhibitors such as aspirin, dipyridamole, ticlopidine, prostacyclin and its derivatives, cell proliferation inhibitors such as ketanserin, corticosteroids, calcium channel blockers, angiotensin converting enzyme inhibitors, angiotensin II receptor antagonists, and antilipidemics such as eicosapentaenoic acid, and lovastatin.

The active agents may be made up in a solid form (including granules, powders or suppositories) or in a liquid form (e.g., solutions, suspensions, or emulsions), and may be subjected to conventional pharmaceutical operations such as sterilization and/or may contain conventional adjuvants, such as stabilizers, wetting agents, emulsifiers, preservatives, cosolvents, suspending agents, viscosity enhancing agents, ionic strength and osmolality adjustors and other excipients in addition to buffering agents. Suitable water soluble preservatives which may be employed in the drug delivery vehicle include sodium bisulfite, sodium thiosulfate, ascorbate, benzalkonium chloride, chlorobutanol, thimerosal, phenylmercuric borate, parabens, benzyl alcohol, phenylethanol or antioxidants such as Vitamin E and tocopherol and chelators such as EDTA and EGTA. These agents may be present, generally, in amounts of about 0.001% to about 5% by weight and, preferably, in the amount of about 0.01 to about 2% by weight.

For administration, the active agents are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. AIternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

A large variety of alternatives are known in the art as suitable for purposes of sustained release and are contemplated as within the scope of the present invention. Suitable delivery vehicles include, but are not limited to, the following: microcapsules or microspheres; liposomes and other lipid-based release systems; crystalloid and viscous instillates; absorbable and/or biodegradable mechanical barriers; and polymeric delivery materials, such as polyethylene oxide/polypropylene oxide block copolymers (e.g. poloxamers), poly-orthoesters, cross-linked polyvinyl alcohol, polyanhydrides, polymethacrylate and polymethacryladmide hydrogels, anionic carbohydrate polymers, etc. Useful delivery systems are well known in the art and are described in, e.g., U.S. Pat. No. 4,937,254, the entire disclosure of which is hereby incorporated by reference.

For use in treating or preventing restenosis, the active agents may be administered by any suitable route, including local delivery, parentally, transdermally, or dermally in dosage unit formulations containing conventional pharmaceutically acceptable carriers, adjuvants, and vehicles. The term parenteral as used herein includes, subcutaneous, intravenous, intramuscular, intrasternal, intratendinous, intraspinal, intracranial, intrathoracic, infusion techniques or intraperitoneally.

Local delivery of the active agents of the invention can be by a variety of techniques that administer the agent at or near the traumatized vascular site. Examples of site-specific or targeted local delivery techniques are not intended to be limiting but to be illustrative of the techniques available. Examples include local delivery catheters, such as an infusion catheter, an indwelling catheter, or a needle catheter, stets, synthetic grafts, adventitial wraps, shunts and stents or other implantable devices, site specific carriers, direct injection, or direct applications. (U.S. Pat. No. 5,981,568, incorporated by reference herein in its entirety.)

Local delivery by an implant describes the surgical placement of a matrix that contains the active agent into the lesion or traumatized area. The implanted matrix can release the active agent by diffusion, chemical reaction, or solvent activators. See, for example, Lange, Science, 249, 1527 (1990).

An example of targeted local delivery by an implant is the use of a stent, which is designed to mechanically prevent the collapse and re-occlusion of the coronary arteries or other vessels. Incorporation of an active agent into the stent permits delivery of the active agent directly to the lesion. Local delivery of agents by this technique is described in Koh, Pharmaceutical Technology (October, 1990). For example, a metallic, plastic or biodegradable intravascular stent is employed which comprises the active agent. The stent may comprise a biodegradable coating, a porous or a permeable non-biodegradable coating, or a biodegradable or non-biodegradable membrane or synthetic graft sheath-like coating, e.g., PTFE, comprising the active agent. AIternatively, a biodegradable stent may also have the active agent impregnated therein, i.e., in the stent matrix.

A biodegradable stent with the active agent impregnated therein can be further coated with a biodegradable coating or with a porous non-biodegradable coating having a sustained release-dosage form of the active agent dispersed therein. This stent can provide a differential release rate of the active agent, i.e., there can be an initial faster release rate of the active agent from the coating, followed by delayed release of the active agent impregnated in the stent matrix, upon degradation of the stent matrix. The intravascular stent also provides a mechanical means of providing an increase in luminal area of a vessel.

Furthermore, the placement of intravascular stents the active agent can also reduce or prevent intimal proliferation. This inhibition of intimal smooth muscle cells and stroma produced by the smooth muscle and pericytes can lead to a more rapid and complete re-endothelization following the placement of the vascular stent. The increased rate of re-endothelization and stabilization of the vessel wall following stent placement can reduce the loss of luminal area and decreased blood flow due to vascular smooth muscle cell proliferation which is one of the primary causes of vascular stent failures.

Another example of targeted local delivery by an implant is the use of an adventitial wrap. The wrap comprises a pharmaceutically acceptable carrier matrix, including but not limited to a Pluronic gel which is free, or contained by a collagen mesh, which gel has dispersed therein the active agent.

Another embodiment of the invention is the incorporation of the active agent into the expanded nodal spaces of a PTFE (Impra, Inc., Tempe, Ariz.) vascular graft-like membrane which can surround, or be placed on the interior or on the exterior surface of, an interlumenal vascular stent, which comprises metal or a biodegradable or nonbiodegradable polymer. The active agent, or a sustained release dosage form of the active agent, fills the nodal spaces of the PTFE membrane wall and/or coats the inner and/or outer surfaces of the membrane.

An active agent may also be coated onto the exterior of the wrap. The wrap and/or the coating is preferably biodegradable.

Another example is a delivery system in which a polymer that contains the active agent is injected into the area of the lesion in liquid form. The polymer then solidifies or cures to form the implant which is retained in situ. This technique is described in PCT WO 90/03768 (Donn, Apr. 19, 1990), the disclosure of which is incorporated by reference herein.

Another example is the delivery of the active agent by polymeric endoluminal sealing. This technique uses a catheter to apply a polymeric implant to the interior surface of the lumen. The active agent incorporated into the biodegradable polymer implant and is thereby released at the surgical site. This technique is described in PCT WO 90/01969 (Schindler, Aug. 23, 1989), the disclosure of which is incorporated by reference herein.

Yet another example of local delivery is by direct injection of vesicles or microparticles into the lesion or artery wall adjacent to the lesion. These microparticles may be composed of substances such as proteins, lipids, carbohydrates or synthetic polymers. These microparticles have the active agent incorporated throughout the microparticle or onto the microparticle as a coating. Delivery systems incorporating microparticles are described in Lange, Science, 249,1527 (1990) and Mathiowitz et al., J. App. Poly. Sci., 26, 809 (1981).

For topical administration, the active agents may be formulated as is known in the art for direct application to a target area. Conventional forms for this purpose include wound dressings, coated bandages or other polymer coverings, ointments, lotions, pastes, jellies, sprays, and aerosols. The percent by weight of the active agent of the invention present in a topical formulation will depend on various factors, but generally will be from 0.005% to 95% of the total weight of the formulation, and typically 1-25% by weight.

The dosage regimen for treating or preventing conditions associate with smooth muscle cell proliferation with the active agents is based on a variety of factors, including the age, weight, sex, medical condition of the individual, the severity of the condition, the route of administration, and the particular compound employed. Thus, the dosage regimen may vary widely, but can be determined by a physician using standard methods. Dosage levels of the order of between 0.1 ng/kg and 10 mg/kg of the active agents per body weight are useful for all methods of use disclosed herein.

In another aspect of the invention, pharmaceutical compositions are provided that comprise an amount effective to treat or prevent conditions associated with smooth muscle cell proliferation of one or more of the active agents of the invention in combination with an amount effective to treat or prevent such a condition of anticoagulants, platelet aggregation inhibitors, smooth muscle cell proliferation inhibitors, calcium channel blockers, angiotensin converting enzyme inhibitors, angiotensin II receptor antagonists, and antilipidemics.

The present invention, by providing methods for treating or preventing conditions associated with smooth muscle cell proliferation is broadly useful to treat or inhibit a diminution in vessel lumen volume, area and/or diameter associated with a procedural vascular trauma. It will be recognized that the active agents and dosage forms (both free and sustained release) of the invention are not restricted in use for therapy following angioplasty; rather, the usefulness of the active agents and dosage forms will be proscribed by their ability to inhibit cellular activities of smooth muscle cells and pericytes in the vascular wall. Thus, other aspects of the invention include therapeutic conjugates and dosage forms and protocols useful in early therapeutic intervention for reducing, delaying, or eliminating (and even reversing) atherosclerotic plaques and areas of vascular wall hypertrophy and/or hyperplasia.

Example 1: Inhibition of Human Smooth Muscle Cell Proliferation

Human coronary artery smooth muscle cells were obtained from Clonetics (San Diego, Calif.) and thawed in a 37° C. water bath for approximately 3 minutes. The cells from one vial were then placed in 40 ml of smooth muscle cell growth medium (Smooth muscle cell basal medium supplemented with 5% fetal calf serum, 0.5 ml/500 ml human epidermal growth factor, 0.5 ml/500 ml gentamycin sulfate, 1 ml/500 ml human fibroblast growth factor beta and 0.5 ml/500 ml Insulin; Clonetics). The cells were divided (20 ml per flask) into two 75 cm² tissue culture flasks. The cells were placed in an incubator at 37° C. in a humidified atmosphere of 5% CO₂ in air. The medium was changed the next day to remove freezing solvents and every 48 hours thereafter. When the culture reached approximately 40% confluence, the volume of medium was increased to approximately 30 ml per flask.

When needed for cell harvest, the flask was rinsed with HEPES, and the cells were removed from the tissue culture flask with trypsin EDTA (Clonetics, San Diego). Nine ml of trypsin was added to each flask and the flask was left at room temperature for 1 minute. Thereafter, the cells were observed under phase contrast microscope until the majority of cells were rounded (approximately 3 minutes). The trypsin was then neutralized with medium containing serum. The cells were collected by centrifugation for 5 minutes at 220 g. The supernatant was removed, the cells were resuspended with 10 ml of smooth muscle cell growth medium and the cell number established by evaluation with a hematocytometer and light microscopy. The cells were diluted to 1×10⁴ cells/ml.

One hundred μl of the cell suspension were aliquoted per well of 96 well plates and allowed to adhere for 24 hours in an incubator at 37° C. in a humidified atmosphere of 5% C02 in air. Thereafter, peptides were added to the wells at concentrations ranging from 0.1 μg/ml to 10 μg/ml. On days 1 and 4, the number of cells per well were counted, and the rate of smooth muscle cell growth was determined.

Several peptides decreased the rate of smooth muscle cell proliferation compared with wells that did not contain peptide (Table 3). These data are represented as the percentage of the control growth rate. Values of less that 100 represent an inhibition of cellular proliferation. AII and AIII (A(2-8)) increased the proliferation of human smooth muscle cells. However, smaller fragments (A(3-8), A(5-8), A(2-7), A(1-6), A(1-6), A(3-7)), and analogues of A(1-7) (1GD, 5GD and 8GD) inhibited smooth muscle cell proliferation.

It is to be understood that the invention is not to be limited to the exact details of operation, or to the exact compounds, compositions, methods, procedures or embodiments shown and described, as obvious modifications and equivalents will be apparent to one skilled in the art, and the invention is therefore to be limited only by the full scope of the appended claims.

TABLE 3
Effect of Angiotensin Peptides on the Proliferation of Human Smooth
Muscle Cells.

| | % Control Proliferation | % Control Proliferation |
|---|---|---|
| Peptide | Day 1 vs Day 4 | Day 1 vs Day 7 |
| AII | 121.4 | 154.5 |
| AIII (A(2-8)) | 121.6 | 136.4 |
| A(1-7) | 73.3 | 63.3 |
| A(3-8) | 74.7 | 61.7 |
| A(5-8) | 63.1 | 55.0 |
| A(2-7) | 82.4 | 64.8 |
| A(1-6) | 76.5 | 61.1 |
| A(1-5) | 85.9 | 83.1 |
| A(3-7) | 80.5 | 70.0 |
| 1GD (Ala4-A(1-7)) | 55.4 | 54.2 |
| 5GD (Lys3-A(1-7)) | 86.6 | 80.1 |
| 8GD (HydroxyPro3- | 72.6 | 66.4 |
| A(1-7)) | | |

We claim

1. A method for inhibiting smooth muscle cell proliferation, comprising contacting smooth muscle cells with an amount effective for inhibiting smooth muscle cell proliferation of at least one active agent comprising a sequence of at least three contiguous amino acids of groups R¹—R⁸ in the sequence of general formula I
R¹—R²—R³—R⁴—R⁵—R⁶—R⁷—R⁸
wherein R¹ is selected from the group consisting of H, Asp, Glu, Asn, Acpc (1-aminocyclopentane carboxylic acid), AIa, Me²Gly, Pro, Bet, Glu(NH₂), Gly, Asp(NH₂) and Suc,
R² is selected from the group consisting of Arg, Lys, AIa, Om, Ser(Ac), Sar, D-Arg and D-Lys;
R³ is selected from the group consisting of Val, AIa, Leu, Lys, norLeu, Ile, Gly, Pro, Hydroxy-Pro, Aib, Acpc and Tyr;
R⁴ is selected from the group consisting of Tyr, Tyr(PO₃)₂, Thr, Ser, AIa, homoSer and azaTyr;
R⁵ is selected from the group consisting of Ile, AIa, Leu, norLeu, Val and Gly;
R⁶ is selected from the group consisting of His, Arg or 6-NH₂-Phe;
R⁷ is selected from the group consisting of Pro or AIa; and
R⁸ is selected from the group consisting of phe, phe(br), ile and tyr,
excluding sequences including R⁴ as a terminal tyr group; and
wherein the active agent does not consist essentially of SEQ ID NO:1, SEQ ID NO:2, or SEQ ID NO:4.

2. The method of claim 1 wherein the active agent comprises a sequence of at least four contiguous amino acids of groups R¹—R⁸ in the sequence of general formula I.

3. The method of claim 1 wherein the active agent comprises a sequence of at least five contiguous amino acids of groups R¹—R⁸ in the sequence of general formula I.

4. The method of claim 1 wherein the active agent comprises a sequence of at least six contiguous amino acids of groups R¹—R⁸ in the sequence of general formula I.

5. The method of claim 1 wherein the active agent comprises a sequence of at least seven contiguous amino acids of groups R¹—R⁸ in the sequence of general formula I.

6. The method of claim 1 wherein the active agent consists essentially of a sequence of at least three contiguous amino acids of groups R¹—R⁸ in the sequence of general formula I.

7. The method of claim 1 wherein the active agent consists essentially of a sequence of at least four contiguous amino acids of groups R¹—R⁸ in the sequence of general formula I.

8. The method of claim 1 wherein the active agent consists essentially of a sequence of at least five contiguous amino acids of groups R¹—R⁸ in the sequence of general formula I.

9. The method of claim 1 wherein the active agent consists essentially of a sequence of at least six contiguous amino acids of groups R¹—R⁸ in the sequence of general formula I.

10. The method of claim 1 wherein the active agent consists essentially of a sequence of at least seven contiguous amino acids of groups R¹—R⁸ in the sequence of general formula I.

11. The method of claim 1 wherein the active agent comprises a sequence selected from the group consisting of angiotensinogen, SEQ ID NO:3, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO: 34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, and SEQ ID NO:51.

12. The method of claim 1 wherein the active agent comprises an amino acid sequence selected from the group consisting of SEQ ID NO:3, SEQ ID NO:5, SEQ ID NO:6, SEQ ID NO:7, SEQ ID NO:8, SEQ ID NO:9, SEQ ID NO:38, SEQ ID NO:40, SEQ ID NO:41, and SEQ ID NO:51.

13. The method of claim 1 wherein the active agent consists essentially of a sequence selected from the group consisting of angiotensinogen, SEQ ID NO:3, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, and SEQ ID NO:51.

14. The method of claim 1 wherein the active agent consists essentially of an amino acid sequence selected from the group consisting of SEQ ID NO:3, SEQ ID NO:5, SEQ ID NO:6, SEQ ID NO:7, SEQ ID NO:8, SEQ ID NO:9, SEQ ID NO:38, SEQ ID NO:40, SEQ ID NO:41, and SEQ ID NO:51.

15. A method for treating or preventing a disorder associated with smooth muscle cell proliferation, comprising administering to a subject in need thereof an amount effective for treating or preventing a disorder associated with smooth muscle cell proliferation of at least one active agent comprising a sequence of at least three contiguous amino acids of groups R¹—R⁸ in the sequence of general formula I
R¹—R²—R³—R⁴—R⁵—R⁶—R⁷—R⁸
wherein R¹ is selected from the group consisting of H, Asp, Glu, Asn, Acpc (1-aminocyclopentane carboxylic acid), AIa, Me²Gly, Pro, Bet, Glu(NH₂), Gly, Asp(NH₂) and Suc,
R² is selected from the group consisting of Arg, Lys, AIa, Om, Ser(Ac), Sar, D-Arg and D-Lys;
R³ is selected from the group consisting of Val, AIa, Leu, Lys, norLeu, Ile, Gly, Pro, Hydroxy-Pro, Aib, Acpc and Tyr;
R⁴ is selected from the group consisting of Tyr, Tyr(PO₃)₂, Thr, Ser, AIa, homoSer and azaTyr;
R⁵ is selected from the group consisting of Ile, AIa, Leu, norLeu, Val and Gly;
R⁶ is selected from the group consisting of His, Arg or 6-NH₂-Phe;
R⁷ is selected from the group consisting of Pro or AIa; and
R⁸ is selected from the group consisting of Phe, Phe(Br), Ile and Tyr, excluding sequences including R⁴ as a terminal Tyr group; and
wherein the active agent does not consist essentially of SEQ ID NO:1, SEQ ID NO:2, or SEQ ID NO:4.

16. The method of claim 15 wherein the active agent comprises a sequence of at least four contiguous amino acids of groups R¹—R⁸ in the sequence of general formula I.

17. The method of claim 15 wherein the active agent comprises a sequence of at least five contiguous amino acids of groups R¹—R⁸ in the sequence of general formula I.

18. The method of claim 15 wherein the active agent comprises a sequence of at least six contiguous amino acids of groups R¹—R⁸ in the sequence of general formula I.

19. The method of claim 15 wherein the active agent comprises a sequence of at least seven contiguous amino acids of groups R¹R⁸ in the sequence of general formula I.

20. The method of claim 15 wherein the active agent consists essentially of a sequence of at least three contiguous amino acids of groups R¹—R⁸ in the sequence of general formula I.

21. The method of claim 15 wherein the active agent consists essentially of a sequence of at least four contiguous amino acids of groups R¹—R⁸ in the sequence of general formula I.

22. The method of claim 15 wherein the active agent consists essentially of a sequence of at least five contiguous amino acids of groups R¹—R⁸ in the sequence of general formula I.

23. The method of claim 15 wherein the active agent consists essentially of a sequence of at least six contiguous amino acids of groups R¹—R⁸ in the sequence of general formula I.

24. The method of claim 15 wherein the active agent consists essentially of a sequence of at least seven contiguous amino acids of groups R¹—R⁸ in the sequence of general formula I.

25. The method of claim 15 wherein the active agent comprises a sequence selected from the group consisting of angiotensinogen, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID N:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, and SEQ ID NO:51.

26. The method of claim 15 wherein the active agent comprises an amino acid sequence selected from the group consisting of SEQ ID NO:3, SEQ ID NO:5, SEQ ID NO:6, SEQ ID NO:7, SEQ ID NO:8, SEQ ID NO:9, SEQ ID NO:38, SEQ ID NO:40, SEQ ID NO:41, and SEQ ID NO:51.

27. The method of claim 15 wherein the active agent consists essentially of a sequence selected from the group consisting of angiotensinogen, SEQ ID NO:3, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34; SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, and SEQ ID NO:51.

28. The method of claim 15 wherein the active agent consists essentially of an amino acid sequence selected from the group consisting of SEQ ID NO:3, SEQ ID NO:5, SEQ ID NO:6, SEQ ID NO:7, SEQ ID NO:8, SEQ ID NO:9, SEQ ID NO:38, SEQ ID NO:40, SEQ ID NO:41, and SEQ ID NO:51.

29. The method of claim 15 wherein the disorder associated with smooth muscle cell proliferation is selected from the group consisting of restenosis, atherosclerosis, vascular wall hypertrophy, and vascular wall hyperplasia.
